(19) United States
(12) Patent Application Publication
(10) Pub. No.: US 2009/0208583 A1
(43) Pub. Date: Aug. 20, 2009

(54) TABLETS COMPRISING CANDESARTAN CILEXETIL

(75) Inventors: Tillmann Rohrich, Rheinfelden (CH); Waltraud Bueb, Zofingen (CH); Enno Schweinberger, Zofingen (CH)

Correspondence Address: HOFFMANN & BARON, LLP, 6900 JERICHO TURNPIKE, SYOSSET, NY, 11791, US

(73) Assignee: Siegfried Generics International AG, Zofingen (CH)

(21) Appl. No.: 12/305,283

(22) PCT Filed: Jun. 14, 2007
(86) PCT No.: PCT/EP2007/005225

§ 371 Date: Dec. 17, 2008

(30) Foreign Application Priority Data
Jun. 20, 2006 (CH) 00998/06

(51) Int. Cl. A61K 9/36; A61K 9/28; A61K 31/41; A61K 31/5415; A61K 9/32; A61P 9/12

(52) U.S. Cl. 424/495; 424/490; 514/381; 514/223.5; 424/497; 424/494

(57) ABSTRACT

Composition in the form of a tablet, comprising candesartan cilexetil and optionally further active agents and usual adjuncts, wherein the surface of at least the active agent candesartan cilexetil in this composition is provided with a coating, made from a compound or a mixture of compounds selected from tri-(C₁-C₆)alkyl citrate, di-(C₁-C₆)alkyl phthalate, di-(C₁-C₆)alkyl sebacate and polydimethysiloxanes and methods for production thereof.

The present invention relates to a solid pharmaceutically active composition in the form of tablets comprising candesartan cilexetil for oral administration.

The compound candesartan cilexetil corresponds to the chemical formula

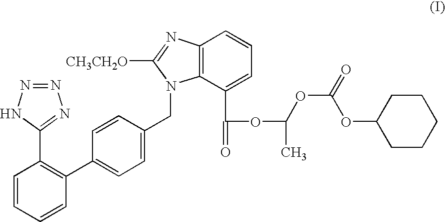

The chemical name of candesartan cilexetil is (+)-1-[[(cyclohexyloxy)carbonyl]oxy]ethyl-2-ethoxy-1-[[2′-(1H-tetrazol-5-yl)-[1,1′-biphenyl]-4-yl]methyl]-1H-benzimidazole-7-carboxylate. Candesartan cilexetil is an angiotensin II receptor antagonist. Candesartan cilexetil is used as a precursor of candesartan, it being hydrolysed to candesartan in the body after intake.

Candesartan cilexetil as an active compound in tablet form is comparatively unstable and forms undesirable by-products, especially during long-term storage. Thus, for example, desethyl-candesartan and related compounds are formed, these reducing the content of active compound in the tablet. This decomposition can often also be attributed to the additives used in the tablet, although this is difficult to determine.

It has now been found that candesartan cilexetil can be processed or pressed with conventional additives to give solid stable compositions in the form of tablets if the composition comprises candesartan cilexetil and optionally further active compounds, as well as conventional additives, and in this composition the surface at least of the active compound candesartan cilexetil is provided with a coating comprising a compound or a mixture of compounds chosen from tri(C₁-C₆)alkyl citrate, di(C₁-C₆)alkyl phthalate, di(C₁-C₆)alkyl sebacate and polydimethylsiloxanes, wherein this compound or the mixture of these compounds by itself forms the coating or represents a substantial constituent of the coating.

It is essential to the invention that the surface of the active compound candesartan cilexetil is provided with the coating according to the invention before the pressing. The active compound here can be provided by itself or in a mixture with any additives, with the coating according to the invention. The active compound and further active compounds possibly present are preferably present in a fine-grained form prior to being coated with the coating. The composition can optionally be granulated prior to being pressed.

A compound chosen from tri(C₁-C₆)alkyl citrate, di(C₁-C₆)alkyl phthalate, di(C₁-C₆)alkyl sebacate and polydimethylsiloxanes having a viscosity in the range of 20-1,300 cSt, or a mixture of these compounds, is preferably used for the coating operation. Surprisingly; this compound or a mixture of these compounds stabilizes the active compound both in the starting mixture and in the tablet, so that scarcely any decomposition phenomena occur even after a relatively long storage time.

The present invention is defined in the claims. In particular, the invention relates to a pharmaceutically active composition in the form of a tablet which comprises candesartan cilexetil and optionally further active compounds, as well as conventional additives, characterizes in that in this composition the surface at least of the active compound candesartan cilexetil is provided with a coating comprising a compound or a mixture of compounds chosen from tri(C₁-C₆)alkyl citrate, di(C₁-C₆)alkyl phthalate, di(C₁-C₆)alkyl sebacate and polydimethyl-siloxanes, wherein this compound or the mixture of these compounds by itself forms the coating or represents a substantial constituent of the coating.

The present invention also relates to the starting mixture comprising the active compound candesartan cilexetil, optionally further active compounds, as well as conventional additives, characterized in that in this starting mixture the surface at least of the active compound candesartan cilexetil is provided with the coating according to the invention.

The starting mixture can be granulated prior to being pressed. In this context, the invention also relates to granules which are suitable for the preparation of the tablet according to the invention, characterized in that these granules have been prepared by granulation of the starting mixture according to the invention. The starting mixture is preferably present in a fine-grained form prior to being pressed.

In this context, the present invention relates to a tablet which comprises the starting mixture according to the invention and/or the granules according to the invention in a pressed form, wherein the surface at least of the active compound candesartan cilexetil is provided with a coating according to the invention.

The present invention also relates to a process for the preparation of the tablet according to the invention.

The present invention also relates to the use of the composition according to the invention, or tablet for oral administration, as an angiotensin II receptor antagonist, in particular for treatment of high blood pressure.

Various polymorphic structures of candesartan cilexetil are known. The present invention is applicable to all of these structures.

Candesartan cilexetil and further active compounds possibly present are preferably present in the tablet in a finely divided form, preferably in a grain size distribution in which about 90% of the grains have an average diameter of less than 20 microns (D₉₀<20 μm) and preferably about 50% of the grains have an aver-age diameter of less than 10 microns (D₅₀<10 μm).

The composition according to the invention preferably comprises the compound having a stabilizing action or the mixture of compounds having a stabilizing action in a concentration in the range of from about 2.0 wt. % to about 45 wt. %; preferably in the range of from about 5 wt. % to about 40 wt. %, based on the weight of the candesartan cilexetil present. The concentrations are as a rule not critical and can be optimized by the person skilled in the art from case to case. Thus, 0.80 mg of triethyl citrate are already sufficient for a tablet which comprises 16 mg of active compound.

The composition according to the invention or the tablet preferably comprises the compound having a stabilizing action or the mixture of compounds having a stabilizing action in a concentration in the range of from about 0.2° wt. % to about 5.0 wt. %, preferably about 0.5 wt. %, based on the weight of the composition or on the tablet weight.

Hydrochlorothiazide (CAS no. [58-93-5] in particular is possible as “further active compounds”, the weight ratio of candesartan cilexetil to hydrochlorothiazide preferably being in the range of from 3:1 to 1:3, preferably in the range of from 2:1 to 1:2 and in particular at about 1.3:1 to 1:1.6. Hydrochlorothiazide acts as a diuretic. Other diuretics are also possible as further active compounds.

In the starting mixture, the surface at least of the active compound candesartan cilexetil is provided with a coating comprising a compound or a mixture of compounds chosen from tri(C₁-C₆)alkyl citrate, di(C₁-C₆)alkyl phthalate, di(C₁-C₆)alkyl sebacate and polydimethylsiloxanes, wherein this compound or the mixture of these compounds by itself forms the coating or represents a substantial constituent of the coating. The term “substantial constituent” means that this compound or the mixture of these compounds represents at least 40 wt. %, preferably at least 50 wt. %, preferably at least 80 wt. % and preferably at least 90 wt. % of the total weight of the coating.

Tri(C₁-C₆)alkyl citrates are, for example, trimethyl citrate, triethyl citrate, tripropyl citrate and tributyl citrate, preferably triethyl citrate, tripropyl citrate and tributyl citrate, preferably triethyl citrate and tributyl citrate, preferably triethyl citrate.

Di(C₁-C₁₀)alkyl phthalates are, for example, dimethyl phthalate, diethyl phthalate, dipropyl phthalate and dibutyl phthalate, preferably dimethyl phthalate, diethyl phthalate and dibutyl phthalate, preferably dimethyl phthalate and diethyl phthalate.

Di(C₁-C₆)alkyl sebacates are, for example, dimethyl sebacate, diethyl sebacate, dipropyl sebacate and dibutyl sebacate, preferably dimethyl sebacate, diethyl sebacate and dibutyl sebacate, preferably diethyl sebacate and dibutyl sebacate, preferably dibutyl sebacate.

The polydimethylsiloxane is preferably a liquid compound having a viscosity preferably in the range of 20-1,300 cSt. The polydimethylsiloxane is preferably a linear polydimethylsiloxane having a degree of polymerization (n) in the range of n=20-400.

Candesartan cilexetil is present in the composition, per tablet, in an amount of from 2 mg to 50 mg, for example in an mount of 2 mg, 4 mg, 8 mg, 10 mg, 16 mg. 24 mg, 32 mg or 45 ma. The content of the active compound in the total weight of the tablet is about 1 wt. % to about 20 wt. %.

The composition according to the invention can comprise as pharmaceutically usable “conventional additives” (excipients) in particular fillers, binders, lubricants, disintegrants (disintegrating agents), dyestuffs and flavour substances. The additives make up the total weight of the composition to 100 wt. %. The qualitative and quantitative suitability and use of these auxiliary substances is known to the person skilled in the art, it being possible for the ratio of the individual additives to one another to be optimized by the person skilled in the art in a manner known per se.

Fillers are, or example, starches, in particular pregelatinized starches, maize starch celluloses, lactose monohydrate, sugars, sucrose, glucose, fructose, sorbitol, microcrystalline cellulose, dextrin, dextrose, mannitol, sorbitol, calcium salts, such as calcium carbonate, calcium sulfate, kaolin, and mixtures thereof.

Binders are, for example, methylcellulose, hydroxypropylcellulose (HPC), polyvinylpyrrolidone, gelatines, gum arabic, ethylcellulose, polyvinyl alcohol, tragacanth, sodium alginate, propylene glycols and mixtures of these compounds.

Lubricants are, for example, magnesium stearates, colloidal silica, calciums stearate, talc, hydrogenated castor oil, microcrystalline wax, beeswax, polyethylene glycol, sodium stearyl fumarate and mixtures of these compounds. These often also act as mould release agents. Pharmaceutically approved dyestuffs and flavour substance are known and are employed in the amounts known per se.

Disintegrants (disintegrating agents) are, for example, calcium carboxymethylcellulose, colloidal silicon dioxide, starch, sodium croscarmellose, crospovidone, sodium starch glycollate and mixtures of these compounds.

A possible embodiment for a process for the preparation of the composition according to the invention is characterized in that candesartan cilexetil is coated with at least one compound having a stabilizing action, and is intensively mixed optionally with at least one or more further active compounds, as well as with at least one additive, preferably with at least one filler and at least one binder, preferably also with at least one lubricant and a disintegrating agent, and the mixture obtained is pressed to a tablet. It is also possible here for all the additives to be coated with a compound having a stabilizing action.

A further embodiment for the process according to the invention comprises a procedure in which candesartan cilexetil is intensively mixed or coated with at least one compound having a stabilizing action, and is mixed optionally with one or more further active compounds, as well as with at least one additive, preferably with at least one filler and at least one binder, the mixture is granulated in the presence of water, the granules obtained are dried and the dry granules are preferably mixed with at least one lubricant and with at least one disintegrating agent and the mixture is pressed to a tablet. It is possible here for all the additives to be coated with a compound having a stabilizing action. The moist granulation known per se is preferably used for the granulation, this process being carried out in a granulator known per se.

A further process for the preparation of the composition according to the invention comprises a procedure in which, in a fluidized bed process, (i) candesartan cilexetil, optionally in a mixture with a further active compound (e.g. hydrochlorothiazide) or further active compounds, together with at least one filler and optionally a disintegrating agent (e.g. lactose monohydrate and/or maize starch), is fluidized, and (ii) a compound having a stabilizing action, dissolved or emulsified in water (e.g. triethyl citrate in water), is added to this fluidized mixture, the mixture present being coated with the compound having a stabilizing action, optionally (iii) the mixture in the fluidized bed is granulated with binder solution (e.g. hyprolose) in a known manner, the mixture obtained (as a powder or granules) is dried and the dry mixture is removed from the fluidized bed, sieved and deagglomerated, and (iv) a disintegrating agent (e.g. Ac-Di-Sol) and a lubricant (e.g. magnesium stearate) is added to the mixture and, after uniform distribution of the components, the mixture obtained is pressed to tablets.

A further process for the preparation of the composition according to the invention comprises a procedure in which (i) candesartan cilexetil, optionally in a mixture with a further active compound (e.g. hydrochlorothiazide) or further active compounds, together with at least one compound having a stabilizing action, is suspended in water and (ii) the suspension, with or without binder (e.g. hydroxypropylcellulose) is sprayed on to one or more carrier substances/fillers (e.g. lactose monohydrate and/or maize starch) in a fluidized bed process; the mixture in the fluidized bed is optionally granulated; the mixture obtained (as a powder or granules) is then dried, (iii) the dry mixture is removed from the fluidized bed, sieved and deagglomerated, (iv) a disintegrating agent (e.g. Ac-Di-Sol) and a lubricant (e.g. magnesium stearate) are added to the mixture and, after uniform distribution of the components, the mixture obtained is pressed to tablets.

A conventional tabletting machine is used for tabletting the mixture, pressures in the range of from 2 KN to 30 KN, preferably 5 KN to 15 KN being used for the tabletting.

The pressed mixture or tablet obtained in such a manner can be provided, as a tablet core, with a coating. Commercially available compounds known per se are preferably used for the preparation of the coating, such as, for example, ethylcellulose, hydroxypropylmethylcellulose (HPMC), hydroxypropylcellulose, methylcellulose, carboxymethylcellulose (CMC), hydroxymethylcellulose (HMC), hydroxyethylcellulose, hydroxypropyl methyl phthalate (HPMP), cellulose acetate, and polyethylene glycols and polymers and copolymers of methacrylic acid which are knolls per se. These are applied to the pressing mixture or the tablet core in a manner known per se from solvents, such as for example, water or methanol, ethanol, isopropyl alcohol or acetone, optionally in a mixture with water. The following examples illustrate the invention.

### Example 1

### Preparation of a Tablet

The additives used in the examples have the following functions:

| Additive: | Function: | Additive: | Function: |
|---|---|---|---|
| triethyl | compound having | hydroxypropyl- | binder |
| citrate, | a stabilizing | cellulose | |
| simethicone | action | (hyprolose) | |
| lactose | filler | croscarmellose | disintegrant |
| monohydrate | | | (disintegrating |
| | | | agent) |
| maize starch | filler/ | magnesium | lubricant/mould |
| | disintegrant | stearate | release agent |

Candesartan cilexetil, lactose monohydrate (water-soluble, e.g. Pharmatose® from DMV.) and maize starch were fluidized in the amounts stated in Table 1 in a fluidized bed apparatus (GPCG 3.1. from Glatt). An aqueous emulsion of the compound Having a stabilizing action [(a) triethyl citrate; (b) simethicone] was fed into the fluidized bed so that the powder mixture was covered with a layer of the compound having a stabilizing action. Thereafter, an aqueous solution of hydroxypropylcellulose (HPC, Klucel®) dissolved in 9 parts of water, were fed in so that granules were obtained. The granules were then dried to equilibrium moisture content in the fluidized bed with the intake air at 60° C., sieved and then mixed with calcium carmellose and magnesium stearate and the mixture was pressed to tablets.

TABLE 1
Candesartan cilexetil formulations

| | Comparison | | |
|---|---|---|---|
| | experiment, | Example 1 (a), | Example 1 (b), |
| | mg | mg | mg |
| Candesartan | 8.0 | 8.0 | 8.0 |
| cilexetil | | | |
| Maize starch | 25.0 | 25.0 | 25.0 |
| Lactose monohydrate | 111.0 | 111.0 | 111.0 |
| Triethyl citrate | —.— | 3.4 | —.— |
| Simethicone | —.— | —.— | 3.4 |
| Klucel (hyprolose) | 5.0 | 5.0 | 5.0 |
| Calcium carmellose | 7.0 | 7.0 | 7.0 |
| Magnesium stearate | 0.6 | 0.6 | 0.6 |
| total | 160.0 | 160.0 | 160.0 |

### Examples 2-5

Example 1 was repeated, but with the compounds and concentrations stated in Table 2. Triethyl citrate was applied to a mixture of the active compound, lactose and maize starch, the mixture was granulated with an aqueous solution of hydroxypropylcellulose, the granules were worked up with the additives listed in Table 2 to give the finished end mixture and this mixture was pressed to tablets with a rotary tablet press (Fette P1). Tablets with two different pressing pressures, or two different hardnesses (50 N and 80 N) were prepared and were stored in open brown glass containers at 50° C. for at least two weeks. At the same time, the end mixture which had not been subjected to pressure was stored at 4° C. in closed brown glass containers for the same period of time as a reference sample for comparison.

TABLE 2

| | Example 2 | Example 3 | Example 4 | Example 5 |
|---|---|---|---|---|
| | mg | mg | mg | mg |
| Active | 8 mg | 8 mg | 8 mg | 8 mg |
| compound/tablet | | | | |
| Composition: | | | | |
| Candesartan | 8.00 | 8.00 | 8.00 | 8.00 |
| cilexetil | | | | |
| Maize starch | 10.50 | 10.50 | 10.50 | 10.50 |
| Lactose monohydrate | 10.50 | 10.50 | 10.50 | 10.50 |
| Triethyl citrate | 0.40 | 0.80 | 1.60 | 3.20 |
| Klucel EXF (binder) | 2.50 | 2.50 | 2.50 | 2.50 |
| Ac-Di-Sol | 7.00 | 7.00 | 7.00 | 7.00 |
| Pharmatose DCL 14 | 120.10 | 120.10 | 120.10 | 120.10 |
| Magnesium stearate | 0.60 | 0.60 | 0.60 | 0.60 |
| total | 159.60 | 160.00 | 160.80 | 162.40 |

Pharmatose DCL 14 = lactose monohyydrate, spray-dried for direct tabletting

### Examples 6-12

Examples 1-5 were repeated, but with the measure that the compositions stated in Table 3 were used.

TABLE 3

| | | | Ex. 8 | Ex. 9 | Ex. 10 | Ex. 11 | Ex. 12 |
|---|---|---|---|---|---|---|---|
| | Ex. 6 mg | Ex. 7 mg | mg | mg | mg | mg | mg |
| Candesartan | 2.00 | 4.00 | 8.00 | 16.00 | 32.00 | 8.00 | 16.00 |
| cilexetil | | | | | | | |
| Hydrochloro- | —.— | —.— | —.— | —.— | —.— | 12.50 | 12.50 |
| thiazide | | | | | | | |
| Maize starch | 10.00 | 10.00 | 10.00 | 10.00 | 20.00 | 10.00 | 10.00 |
| Lactose | 135.80 | 133.80 | 129.80 | 121.80 | 243.60 | 117.30 | 109.30 |
| monohydrate* | | | | | | | |
| Triethyl | 0.80 | 0.80 | 0.80 | 0.80 | 1.60 | 0.80 | 0.80 |
| citrate | | | | | | | |
| Klucel | 6.40 | 6.40 | 6.40 | 6.40 | 12.80 | 6.40 | 6.40 |
| Ac-Di-sol | 4.00 | 4.00 | 4.00 | 4.00 | 8.00 | 4.00 | 4.00 |
| Magnesium | 1.00 | 1.00 | 1.00 | 1.00 | 2.00 | 1.00 | 1.00 |
| stearate | | | | | | | |
| total | 160.00 | 160.00 | 160.00 | 160.00 | 320.00 | 160.00 | 160.00 |

*= pulverulent for granulation

Test Results from Example 1

The tablets prepared in Example 1 were stored in open bottles at 50° C. for two weeks. The contamination profile of the tablets was then determined. A comparison sample stored in a closed bottle for the same time, but at 4° C., was used as a reference.

The contamination profile is characterized by desethyl-candesartan cilexetil as the main impurity, which shows a significant increase during the storage, namely at ET [min] 8.2. The main peak of candesartan cilexetil is at RT [min] 16.5. The results are listed in Table 4.

TABLE 4

| | | Stored at | Stored at | |
|---|---|---|---|---|
| | | 4° C. | 50° C. | Difference |
| | RT [min] | [% (area)] | [% (area)] | [% (area)] |
| Comparison | 6.3 | 0.222 | 0.204 | −0.017 |
| experiment | 8.2 | 0.123 | 0.904 | 0.781 |
| according to | 14.8 | — | 0.201 | 0.210 |
| Example 1 | 16.5 | 99.655 | 97.595 | −2.060 |
| | 19.5 | — | 0.308 | 0.308 |
| | 23.1 | — | 0.242 | 0.242 |
| | 27.9 | — | 0.545 | 0.545 |
| Example 1 (a) | 6.3 | 0.214 | 0.214 | 0.001 |
| | 8.2 | 0.138 | 0.226 | 0.088 |
| | 16.5 | 99.648 | 99.360 | −0.069 |
| Example 1 (b) | 3.4 | 0.245 | 0.123 | −0.122 |
| | 6.3 | 0.221 | 0.215 | −0.006 |
| | 8.2 | 0.245 | 0.275 | 0.030 |
| | 16.5 | 99.351 | 99.386 | 0.035 |

The impurity at RT=6.3 is a by-product associated with the synthesis. The amount remains constant during storage and does not influence the stability and the result.

A critical, compound in the stability test is the increase in desethyl-candesartan cilexetil (RT=8.2), which is a degradation product of candesartan cilexetil. The use of triethyl citrate and dimethicone (CAS No. [9006-65-9], demonstrates the stabilizing action of these compounds for candesartan cilexetil tablets during storage.

Test Results from Examples 2 to 5

The tablets from Examples 9, 3, 4 and 5 were investigated with an amended HPLC method, which led to other retention times. The results obtained are given in Table 5. In this, the peaks correspond to the following compounds:

Peak at 1.7-1.8 min=desethyl-candesartan cilexetil
Peak at 3.8-3.9 min=candesartan cilexetil
Peak at 9.1-9.2 min=N-ethyl-candesartan cilexetil

TABLE 5

| | | | Difference |
|---|---|---|---|
| | | Stored at 50° C. | (50° C.-4° C.) |
| | | [% (area)] | [% (area)] |
| | RT [min] | after 2 weeks | after 2 weeks |
| Example 2 | 1.8 | 0.140 | 0.092 |
| | 3.9 | 99.44 | −0.12 |
| | 9.1 | 0.069 | 0.029 |
| Example 3 | 1.8 | 0.078 | 0.035 |
| | 3.9 | 99.24 | −0.33 |
| | 9.1 | 0.053 | 0.011 |
| Example 4 | 1.8 | 0.077 | 0.031 |
| | 3.9 | 99.42 | −0.14 |
| | 9.1 | 0.055 | 0.016 |
| Example 5 | 1.8 | 0.079 | 0.035 |
| | 3.9 | 99.51 | −0.08 |
| | 9.1 | 0.053 | 0.022 |

The test results clearly show the stabilizing action of triethyl citrate.

Test Results with Further Compounds Having a Stabilizing Action

Analogous results are obtained if, analogously to the preceding examples, the following compounds having a stabilizing action are used instead of triethyl citrate and simethicone: trimethyl citrate, tributyl citrate, dibutyl sebacate, dimethyl phthalate, diethyl phthalate and/or dibutyl phthalate.

1. A composition in the form of a tablet, comprising candesartan cilexetil and optionally further comprising one or more other active compounds, and one or more additives, wherein the surface at least of candesartan cilexetil is provided with a coating comprising a compound chosen from tri(C₁-C₆)alkyl citrate, di(C₁-C₆)alkyl phthalate, di(C₁-C₆)alkyl sebacate, a polydimethylsiloxanes or a mixture of two or more thereof, wherein the compound by itself forms the coating or represents a substantial constituent of the coating.

2. The composition according to claim 1, wherein candesartan cilexetil and optional one or more other active compounds are present in a finely divided form with a grain size distribution in which 90% of grains have an average diameter of less than 20 microns (D₉₀<20 μm) and 50% of grains have an average diameter of less than 10 microns (D₅₀<10 μm).

3. The composition according to claim 1, wherein the compound is present in a concentration in the range of from 2.0 wt. % to about 45 wt. %, based on the weight of candesartan cilexetil present.

4. The composition according claim 1, wherein the compound forms at least 40 wt. % of the total weight of the coating.

5. The composition according to claim 1, wherein the compound is present in a concentration in the range of from 0.2 wt. % to 5.0 wt. %, based on the weight of the composition.

6. The composition according to claim 1, comprising one or more other active compounds wherein one or more other active compounds is hydrochlorothiazide or another diuretic, and wherein the weight ratio of candesartan cilexetil to hydrochlorothiazide is in the range of from 3:1 to 1:3.

7. The composition according to claim 1, wherein tri(C₁-C₆)alkyl citrate is chosen from trimethyl citrate, triethyl citrate, tripropyl citrate and tributyl citrate.

8. The composition according to claim 1, wherein di(C₁-C₆)alkyl phthalate is chosen from dimethyl phthalate, diethyl phthalate, dipropyl phthalate and dibutyl phthalate.

9. The composition according to claim 1, wherein di(C₁-C₆)alkyl sebacate is chosen from dimethyl sebacate, diethyl sebacate, dipropyl sebacate and dibutyl sebacate.

10. The composition according to claim 1, wherein the compound is a polydimethylsiloxane which is a liquid having a viscosity in the range of 20-1,300 cSt, and is a linear polydimethylsiloxane having a degree of polymerization (n) in the range of n=20-400.

11. The composition according to claim 1, wherein candesartan cilexetil is present, per tablet, in an amount of from 2 mg to 50 mg.

12. The composition according to claim 1, wherein one or more additives comprise fillers, binders, lubricants, disintegrating agents, dyestuffs and/or flavour substances.

13. A process for preparing the composition according to claim 1, comprising forming a mixture wherein candesartan cilexetil is coated with at least one compound having a stabilizing action, and is intensively mixed optionally with one or more other active compounds, and with at least one additive, wherein at least one additive is coated with at least one compound having a stabilizing action, and the mixture obtained is pressed to form a tablet.

14. A process for preparing the composition according to claim 1, comprising forming a first mixture wherein candesartan cilexetil is intensively mixed or coated with at least one compound having a stabilizing action, and is mixed optionally with one or more other active compounds, and with at least one additive, wherein at least one additive is coated with a compound having a stabilizing action, the first mixture is granulated in the presence of water to form granules which are dried and mixed with at least one lubricant and at least one disintegrating agent to form a second mixture, and wherein the second mixture is pressed to form a tablet.

15. A process for the preparing the composition according to claim 1, wherein in a fluidized bed, (i) candesartan cilexetil, optionally in a mixture with one or more other active compounds, together with at least one filler and optionally a disintegrating agent, is fluidized to form a first mixture, and (ii) a compound having a stabilizing action, which is dissolved or emulsified in water, is added to the first mixture, and wherein the first mixture is coated with the compound having a stabilizing action to form a second mixture, (iii) the second mixture is optionally granulated with a binder solution in the fluidized bed to form granules, which are dried, removed from the fluidized bed, sieved and deagglomerated, and (iv) a third mixture with uniformly distributed components is formed wherein a disintegrating agent and a lubricant are added to the second mixture optionally granulated with a binder solution, and the third mixture is pressed to form tablets.

16. A process for preparing the composition according to claim 1, wherein (i) candesartan cilexetil, optionally in a mixture with one or more other active compounds, together with at least one compound having a stabilizing action, is suspended in water to form a first mixture which is a suspension and (ii) the suspension, with or without a binder, is sprayed onto one or more carrier substances/fillers in a fluidized bed to form a second mixture, the second mixture in the fluidized bed is optionally granulated, and granules obtained are dried, (iii) granules are removed from the fluidized bed, sieved and deagglomerated, (iv) a third mixture with uniformly distributed components is formed wherein a disintegrating agent and a lubricant are added to the second mixture optionally granulated and, wherein the third mixture is pressed to form tablets.

17. A process according to claim 13, wherein the tablet is provided with a coating comprising ethylcellulose, hydroxypropylmethylcellulose, hydroxypropylcellulose, methylcellulose, carboxymethylcellulose, hydroxymethylcellulose, hydroxyethylcellulose, hydroxypropyl methyl phthalate, cellulose acetate, polyethylene glycols, and polymers and copolymers of methacrylic acid.

18. A starting mixture for preparing the composition according to claim 1, wherein the starting mixture comprises candesartan cilexetil, optionally one or more other active compounds, and one or more additives, and the surface at least of candesartan cilexetil is provided with a coating according to claim 1.

19. Granules which are suitable for the preparation of a composition according to claim 1 comprising candesartan cilexetil, optionally one or more other active compounds, and one or more additives, and the surface at least of candesartan cilexetil is provided with a coating according to claim 1.

20. Granules according to claim 19, prepared by granulation of the starting mixture according to claim 18.

21. A method for treating high blood pressure in a patient comprising administering the composition according to claim 1 to the patient in need thereof.
